SEQUENCE LISTING (3 sequences)

SEQ ID NO: 1 - DNA, 8532 bp, Homo sapiens
atctgcacctcctcatatagggttgatccaagtttcacagacatcactgagttcttagtggactcagctattggggctgttctcacactttttttttctttgcaagaatcagcaatgggtgcaagtggacctgtgtaggacgtccagtgaaacattgtgttggtgaatcagctagaatccatccaagaactcagccagcctggtgtggggtgagatctgatccttgaatgtccctcagtggcttttagggctggcaggttcagaagggccctctcatcacccccccagggcctcattccttgtttaacactttgctatcacagtcttgaatccttgtaattgaacaatggaccccacattttcactttgcactggtttctgattctgtaaccgatcctgtccccctctcttgtctcattcactctgggaattgtccccacattctgagacctttcagcagtgccccaacgaggttcctgcccttatctgaagctccaccctcacccccatggcggcaccgcaggcagccctgcttttgcgtcccgcgtaggcaggctgtgcaccggagtcacgaccccctgattcagcctaggcagccacagcttgactgctcccgccggacaagccctactgtgctatctgccgctcttcccttcctcttcccagggggtccgcgtcaggggaggcgcagctgtgtgcattccgggagcttcagacccccgtgtccagcagctccttcgtttcctgggtgctggggcggccttcccagcgaagagctcaactcagcgggacgtttggaggctctctgccccaaggcgctggggagtgtgcggcgggacagtcgtgcttgcctttttcactttcagagtgtccacgccccacccgtttggtcactgcaggtcagtccagtccagcccggcccaccccaccggtgcgtgtctgtcgcacgtggcagacgccatactctctgttcttgtttaaagcccaggatctactgggccctggaggcaagaggtgaacgcagcggaatccacgctgagctgcccgggaacggagcttccaaccccagaaggaggactctgtgctcctacaccttaaccctttttagcccgaaacttctccaacttccttggctttgtttagagctcgacagcgccgccccctggcgctcgttgtgaggacagtagaggagagaggcaagggtgtttttaaacagtttgcctctcaccattatgggggcgacccgagggggagacccactcttccgcattcccggtaagtgaaccaccggaagaggtcgaaagtgacggattcccatgtcctcctccagccccccccccaccctgcccatccacaggacggtggctcttcagtgccctttgccgagcaagtggcgtttctatgcacgtgggtatcaattcggactctggacgaaatggaaacctccttagccgacccgggtgggatcagctgggatcctgcgcgctcccctggggggttgccagccactctgttggggtgcaagaagcaccatccttcggaagctgggccgaaactggccaggctgactcgctcccacgcgcccgcccctacccggcgccgcagcaattcacctgccaccgcctctgagccgggtccggacttcggcgccctgacagtgtccccgcgacttccccacccgatgagatggggtctggcgttggccagtgcgtgtccagggactcgcgggtccctggccagccatggggcagagggcgctggtgttaggccagtcttccccaccctgccccgtcaccccagccacacccactgtcctgtgaggccaagcgcgctccgctggtttcctgagccaggcaccttggccgcggacaggatccagctgtctctccttgcgatcctgtcttcggggaagtccacgtcctaggcaggtcctcccaaagtgcccttggtgccgatcacccctcccagcgtcttgcaggtcctgtgcaccacctcccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgyccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtrttaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgmgagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgctctcgggttgtgtgtctgttcacttttcctccctcagtgccctcattttatgtcattaaatggggctcacaaaccatgcaaatgctatgagatgcatggagggctgccctgtaccccagcacttgtgttgtctggtgrtggcaccatctctgattttcaaagctttttccagaggctattattttcactgtagaatgatttcatgctatctctgtgtgcacaaatatttattttctttctgtaaccataacaacttcatatatgaggacttgtgtctctgtgcttttaaatgcataaatgcattataggatcatttgttggaatgaattaaataaacccttcctggggcatctggcgaatcccagctgtgtgtccggtgtatggtttggcattatctcctctgcgagatatccaaattcactgtagtcatgaagggtctcagtttgtggctctcattcaaatattcatttctaaacgtctcatccagtatgaaatcattctcatctcttttggagattaacaacatcatcttttcaatgcacacgtttcttgggctcacttttctaaggtggtagggctggctgaatgcaatatgcagggctcggaaagattttttaaagaagaaattaaaagcaagtagagtccaggcaaatattcagatgctttatatgtctggataatgctgaactcatgagttttagtttgactgattattgtgaagaccgggttggagattttgacatccatcgcagaagaagtaatggctttagtgtgtgtgtgtgtgtgtgctggggaagctccatgcacagtgccctatggagataacaagctgagccatgctccccctaagtagcagactaagtctttgtgaaggaagagctacacaaatgggggcaggacaggtgcagataaatggggctgggagaccagaggagacagtgacaccttatagttcgccccctgttacccagccttctgtttgtcaaaagagtctgctcccagtcactgtcaaactgacttgtagggcctcattgcgttaggatttcttcttattccagaagaggggcattttcttaaggaacactggaagaccaaaacacactttcaaaacctagaggcaaaaacccttcatgcagcacttgggccccaggacattagttgtgcggggccctgagcttccctgtcctcctcacttcctgctgcctgggggatcagcagttctgtttataggtctcatctgaacttgagattctcaaaacgctaaatagccatagtgcctctcagggaaagataccaggaccacataaacaaatcagttagctttaaaaactatccctgagcatttaaaatcaggatagaccttgtgaaaccagagccatgggtcaacctgtgtgatctctgctttctgttcacatcattggacatccaggtctgagggagactcccagggaccagttgctggtgaaatttcatagcacaaaagtccggggcaagaaagccaaggtggtatttctggataagccagcattcaagtttgtttgtttgtttgtttgtttgtttttcctagcctgctgttttaaagtaaacagaatgcatttttttaagtcaaatgactttgttattttttttttccagttctcacctatttcttagattagttcagcaattatttactgagcatttactctgtgcctttcatagtgataggcacaatgacaagtccctaccatataagttagactctggcaggggagaaagatgcaaaacaactgatcacccccaaattgtacttaacttagaaacagtgataagtgcaggggaagaaaagcacagcacactctgaaaaggcgcacgaggaaggcaggatttagagtggaggactagagggagcttcctggacaagctgacacttaacaccagacctgaaggggaaggaggggtttgtcaaatgcaaactggaggggaagcagtccaggtgggaaggatcacacctgcaaaggccctgtactgggaagagccctggtggagcggactgggcatagtgaacaaggtgaggtgggctgcaaggcagctgaagaggtggaaagagagatacaagcagtgggagatgactgtaggggctgtaggtcaaagacactgaaaaaaagactgaaagagtgacattgaaaaatgttctgggtgcaagtgggggcactcaaggagttttgatgagagtgcactgggattcaatttatgtactgcattgtttgggaagataacaactacttctagatgtatttacatgtccctcttgggcaggaacctgcacaatttccgctgtaagcaccccgcagggctgatatgtggtgtgaacagcatacacacctgggtgtgaccccagcctgaaacctgctggtcacatggccacgggcaccacatgacccttcaaaggctgt

SEQ ID NO: 2 - DNA, 101 bp, Homo sapiens
ttacttatttttgacagtgttgaaaatgttcagaaggttgctctagattgmgagaagagacaaacacctcccaggagacagttcaagaaagcttcaaactg

SEQ ID NO: 3 - DNA, 511 bp, Homo sapiens
gcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgyccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttc

(19) United States
(12) Patent Application Publication
(10) Pub. No.: US 2007/0281300 A1
(43) Pub. Date: Dec. 6, 2007

(54) Thrombomodulin (Thbd) Haplotypes Predict Outcome

(76) Inventors: James Russell, Vancouver (CA); Keith R. Walley, North Vancouver (CA)

Correspondence Address: McKenna Long & Aldridge, 1900 K Street, NW, Washington, DC, 20006, US

(21) Appl. No.: 10/591,325

(22) PCT Filed: Mar. 4, 2005
(86) PCT No.: PCT/CA05/00356

§ 371 Date: Jun. 27, 2007

Related U.S. Application Data
(60) Provisional application No. 60/549,559, filed on Mar. 4, 2004.

(51) Int. Cl. C12Q 1/68; C07H 21/02; C07H 21/04

(52) U.S. Cl. 435/6; 536/23.5; 536/24.31

(57) ABSTRACT

The invention provides methods and kits for obtaining a prognosis for a subject having or at risk of developing an inflammatory condition. The method generally comprises determining a thrombomodulin genotype(s) of a subject for one or more SNPs, comparing the determined genotype with known genotypes for the polymorphism that correspond with the ability of the subject to recover from the inflammatory condition and identifying subjects based on their prognosis.

### FIELD OF THE INVENTION

The field of the invention relates to the assessment of subjects with an inflammatory condition.

### BACKGROUND OF THE INVENTION

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit. Care Med (1996) 24:381-4; STUBER F. et al. Journal of Inflammation (1996) 46:42-50; and WEYTKAMP J H. et al. Infection (2000) 28:92-6). Furthermore, septic and non-septic stimuli such as bacterial endotoxin and cardiopulmonary bypass (CPB), respectively, activate the coagulation system and trigger a systemic inflammatory response syndrome (SIRS).

Thrombomodulin (THBD) is encoded by an intronless gene. THBD is found on endothelial cell surfaces and forms a high affinity complex with thrombin and inhibits the pro-coagulant activities of thrombin. THBD is an endothelial-specific type I membrane receptor (glycoprotein receptor). The binding of thrombin to THBD results in the activation of protein C and the activated protein C anti-coagulant pathway. Activated protein C binds to protein S and in turn degrades clotting factors Va and VIIIa and reduces the amount of thrombin generated. Activated protein C also binds the endothelial protein C receptor and protein C receptor on leukocytes, initiating intracellular signaling that leads to inhibition of the inflammatory cytokine and adhesion molecule response. Thus, THBD also has anti-inflammatory activity, inhibiting both cytokine formation and leukocyte-endothelial cell adhesion.

The activation of protein C by the THBD-thrombin complex is reduced in sepsis, resulting in perturbations in the coagulation system and disseminated intravascular coagulation. THBD biosynthesis has been shown to be decreased by both endotoxin and hypoxia. Microthrombi generated in this hyper-coagulable state lead to multiple system organ failure.

Systemic inflammatory response syndrome (SIRS) is characterized by increased inflammation (relative to anti-inflammatory processes), increased coagulation (relative to anti-coagulant processes), and decreased fibrinolysis. THBD is an endothelial cell surface receptor which binds to circulating thrombin and inhibits thrombin coagulant activities. The thrombomodulin:thrombin complex activates protein C and also has downstream anti-inflammatory effects.

Protein C, when activated to form activated protein C (APC), plays a major role in three biological processes or conditions: coagulation, fibrinolysis and inflammation. Acute inflammatory states decrease levels of the free form of protein S, which decreases APC function because free protein S is an important co-factor for APC. Sepsis, acute inflammation and cytokines decrease thrombomodulin expression on endothelial cells resulting in decreased APC activity or levels. Septic shock also increases circulating levels of thrombomodulin, which is related to increased cleavage of endothelial cell thrombomodulin. Another mechanism for decreased APC function in sepsis is that endotoxin and cytokines, such as TNF-∝, down-regulate endothelial cell protein C receptor (EPCR) expression, thereby decreasing protein C and APC signaling via EPCR. Severe septic states such as meningococcemia, also result in protein C consumption. Depressed protein C levels correlate with purpura, digital infarction and death in meningococcemia.

Protein C is also altered in non-septic patients following cardiopulmonary bypass (CPB). Total protein C, APC and protein S decrease during CPB. Following aortic unclamping (reperfusion at the end of CPB) protein C is further activated so that the proportion of remaining non-activated protein C is greatly decreased. A decrease of protein C during and after CPB increases the risk of thrombosis, disseminated intravascular coagulation (DIC), organ ischemia and inflammation intra- and post-operatively. Patients who have less activated protein C generally have impaired recovery of cardiac function, consistent with the idea that lower levels of protein C increase the risk of microvascular thrombosis and myocardial ischemia. Aprotinin is a competitive inhibitor of APC, and is sometimes used in cardiac surgery and CPB. Aprotinin has been implicated as a cause of post-operative thrombotic complications after deep hypothermic circulatory arrest.

Septic and non-septic stimuli such as bacterial endotoxin and cardiopulmonary bypass (CPB), activate the coagulation system and trigger a systemic inflammatory response syndrome (SIRS). A decrease in protein C levels have been shown in patients with septic shock (GRIFFIN J H. et al. (1982) Blood 60:261-264; TAYLOR F B. et al. (1987) J. Clin. Invest. 79:918-925; HESSELVIK J F. et al. (1991) Thromb. Haemost. 65:126-129; FIJNVANDRAAT K. et al. (1995) Thromb. Haemost. 73(1): 15-20), with severe infection (HESSELVIK J F. et al. (1991) Thromb. Haemost. 65:126-129) and after major surgery (BLAMEY S L. et al. (1985) Thromb. Haemost. 54:622-625). It has been suggested that this decrease is caused by a decrease in protein C transcription (SPEK C A. et al. J. Biological Chemistry (1995) 270(41):24216-21 at 24221). It has also been demonstrated that endothelial pathways required for protein C activation are impaired in severe menigococcal sepsis (FAUST S N. et al. New Eng. J. Med. (2001) 345:408-416). Low protein C levels in sepsis patients are related to poor prognosis (YAN S B. and DHAINAUT J-F. Critical Care Medicine (2001) 29(7):S69-S74; FISHER C J. and YAN S B. Critical Care Medicine (2000) 28(9 Suppl):S49-S56; VERVLOET M G. et al. Semin Thromb Hemost. (1998) 24(1):33-44; LORENTE J A. et al. Chest (1993) 103(5):1536-42). Recombinant human activated protein C reduces mortality in patients having severe sepsis or septic shock (BERNARD G R. et al. New Eng. J. Med. (2001) 344:699-709). Thus protein C appears to play a role in the systemic inflammatory response syndrome.

The human thrombomodulin sequence maps to chromosome 20p12-cen and extends over 8.5 kb. A representative Homo sapiens thrombomodulin sequence is listed in GenBank under accession number AF495471 (8532 bp).

A number of polymorphisms have been observed in the promoter region (G-201A, G-33A which correspond to positions 2791 and 2388 of SEQ ID NO:1 respectively) and the coding region (F127A, C1418T, and G1456T which correspond to positions 2716, 4007 and 4045 of SEQ ID NO:1 respectively) of the thrombomodulin sequence have been tested for association to the occurrence and risk of thrombotic events and cardiovascular disease (Doggen C J. et al. (1998) Thromb Haemost 80:743-8; Ireland H. et al. (1997) Circulation 96:15-8; Kunz G. et al. (2002) Blood 99:3646-3653; Nakazawa F. T. et al. (2002) Atherosclerosis 164:385-7; and Ohnishi Y T. et al. (2000) Hum Genet. 106:288-92). The −33A allele has been found to decrease promoter activity of the thrombomodulin promoter region and may be associated with altered soluble thrombomodulin serum levels and coronary artery disease, carotid atherosclerosis, and myocardial infarction. The G-201A and G1456T polymorphisms were found to be rare in patients with severe thrombophilia and possibly functionally irrelevant. The G127A polymorphism was weakly associated with increased risk of myocardial infarction in young men when additional risk factors such as smoking were present.

A G-to-A polymorphism at position −33 (2388 of SEQ ID NO:1) in the promoter region of the thrombomodulin gene is particularly frequent in the Asian population. The thrombomodulin G-33A polymorphism is near a consensus sequence for transcription control elements, and reporter gene assays have shown that the −33A allele decreases promoter activity. Interestingly, it has been found that in CAD patients homozygous −33G allele soluble thrombomoduiin levels increased with the extent of CAD. In CAD patients who were homozygous or heterozygous for the −33A allele, levels of soluble thrombomodulin did not change with the extent of vessel disease.

The C1418T (position 4007 of SEQ ID NO:1) polymorphism has been associated with formation of varicose veins. With regards to the risk of myocardial infarction associated with the C1418T polymorphism, prior studies have been inconsistent (Chao et al. (2004) Am J Cardio 93(2):204-207; Park et al. (2002) Hypertens Res 3:389-94; Wu et al. (2001) Circulation 103(10):1386-1389; and Norlund et al. (1997) Thromb Haemost 77(2):248-51). Furthermore, this site was found not to be associated with risk of venous thromboembolism (Faioni et al. (2002) Br J Haematol 118(2):595-9) and not to be associated with risk of late fetal loss (Franchi et al. (2001) Brit J Haematology 114(3):641). The associations of these polymorphisms with various thrombotic events and cardiovascular disease are uncertain and there have been a number of negative studies. Previous studies have not examined the association of thrombomodulin polymorphisms with clinical outcome in critical illness such as systemic inflammatory response syndrome and sepsis.

### SUMMARY OF THE INVENTION

This invention is based in part on the surprising discovery that particular single nucleotide polymorphisms (SNPs) from the human thrombomodulin (THBD) sequence can be predictors of subject outcome from an inflammatory condition.

This invention is based in part on the surprising discovery of thrombomodulin SNPs associated with improved prognosis or subject outcome, in subjects with an inflammatory condition. Furthermore, various THBD SNPs are provided which are useful for subject screening, as an indication of subject outcome, or for prognosis for recovery from an inflammatory condition.

This invention is also based in part on the identification the particular nucleotide at the site of a given SNP which is associated with a decreased likelihood of recovery from an inflammatory condition (i.e. ‘risk genotype’) or an increased likelihood of recovery from an inflammatory condition (i.e. ‘protective genotype’).

In accordance with one aspect of the invention, methods are provided for obtaining a prognosis or predicting ability to recover for a subject having or at risk of developing an inflammatory condition, the method including determining a genotype of the subject which includes one or more polymorphic sites in the subject's THBD sequence, wherein the genotype is indicative of an ability of the subject to recover from the inflammatory condition.

In accordance with another aspect of the invention, methods are provided for obtaining a prognosis or predicting ability to recover for a subject having or at risk of developing an inflammatory condition, the method including the step of determining a haplotype for the subject. The haplotype may correspond to positions 5110, 5318 and 6235 of SEQ ID NO:1. The risk haplotypes represented by 5110G/5318A/6235A, 5110A/5318A/6235A, 5110G/5318A/6235G, or 5110A/5318A/6235G. The protective haplotype represented by 5110A/5318C/6235A. The method may further include the step of obtaining the subject's genetic sequence information prior to determining the haplotype for a subject and furthermore the method may include the step of obtaining a biological sample from the subject containing genetic sequence information. Additionally, the method may comprise identifying a patient at risk of or having an inflammatory condition.

In accordance with another aspect of the invention, methods are provided for obtaining a prognosis or predicting ability to recover for a subject having or at risk of developing an inflammatory condition, the method including the step of determining a genotype of the subject which includes one or more polymorphic sites in the subject's THBD sequence, wherein the genotype is indicative of an ability of the subject to recover from the inflammatory condition. The method may further include the step of obtaining the subject's genetic sequence information prior to determining the genotype for a subject and furthermore the method may include the step of obtaining a biological sample from the subject containing genetic sequence information. Additionally, the method may comprise identifying a patient at risk of or having an inflammatory condition.

In accordance with another aspect of the invention, methods are provided for obtaining a prognosis or predicting ability to recover for a subject having or at risk of developing an inflammatory condition, the method may including any one or more of the following steps:
(a) identifying a patient at risk of or having an inflammatory condition;
(b) obtaining a biological sample from the subject;
(c) obtaining the subject's genetic sequence information;
(d) determining a genotype of the subject which includes one or more polymorphic sites in the subject's THBD sequence;
   wherein the genotype is indicative of an ability of the subject to recover from the inflammatory condition.

The polymorphic site may be at position 5318 of SEQ ID NO:1 or at a polymorphic site in in linkage disequilibrium thereto. Alternatively, the polymorphic site in linkage disequilibrium with position 5318 may correspond to position 4007 of SEQ ID NO:1. The polymorphic site in linkage disequilibrium with position 5318 may have a D′ value of ≧0.8 (or r² value ≧0.8). The method may further include comparing the genotype determined with known genotypes which are known to be indicative of a prognosis for recovery from: (i) the subject's type of inflammatory condition; or (ii) another inflammatory condition. The method may further include determining the thrombomodulin sequence information for the subject and the method may further include determining the genotype from a nucleic acid sample obtained from the subject. Determining of genotype may include one or more of the following: restriction fragment length analysis; sequencing; hybridization; oligonucleotide ligation assay; ligation rolling circle amplification; 5′ nuclease assay; polymerase proofreading methods; allele specific PCR; and reading sequence data.

A risk genotype of the subject may be indicative of a decreased likelihood of recovery from an inflammatory condition or an increased risk of having a poor outcome. Risk genotype where the subject is critically ill may be indicative of a prognosis of severe cardiovascular or respiratory dysfunction. The risk genotype may include at least one A nucleotide at position 5318 or at least one C nucleotide at position 4007 of SEQ D NO:1.

A protective genotype of the subject may be indicative of an increased likelihood of recovery from an inflammatory condition. Where the subject is critically ill the protective genotype may be indicative of a prognosis of less severe cardiovascular or respiratory dysfunction. The protective genotype may be homozygous for the C nucleotide at position 5318 or homozygous for the T nucleotide at position 4007 of SEQ ID NO:1.

In accordance with another aspect of the invention, methods are provided for identifying a polymorphism in a thrombomodulin sequence that correlates with prognosis of recovery from an inflammatory condition in a subject, the method including:
(a) obtaining thrombomodulin sequence information from a group of subjects with an inflammatory condition;
(b) identifying at least one polymorphic nucleotide position in the thrombomodulin sequence in the subjects;
(c) determining a genotype at the polymorphic site for individual subjects in the group;
(d) determining recovery capabilities of individual subjects in the group from the inflammatory condition; and
(e) correlating genotypes determined in step (c) with the recovery capabilities determined in step (d)
thereby identifying said thrombomodulin polymorphisms that correlate with recovery.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. As used herein the term “inflammatory condition” specifically excludes myocardial infarction. In further embodiments congestive heart failure is specifically excluded from inflammatory conditions. In still further embodiments post-pump syndrome is specifically excluded from inflammatory conditions. And in yet further embodiments cardiac stun syndrome is specifically excluded from inflammatory conditions.

The determining of a genotype may be accomplished by any technique known in the art, including but not limited to one or more of: restriction fragment length analysis; sequencing; hybridization; oligonucleotide ligation assay; ligation rolling circle amplification; 5′ nuclease assay; polymerase proofreading methods; allele specific PCR; matrix assisted laser desorption ionization time of flight MALDI-TOF mass spectroscopy micro-sequencing assay; gene chip hybridization assays; and reading sequence data.

In accordance with another aspect of the invention, there is provided a kit for determining a genotype at a defined nucleotide position within a polymorphism site in a thrombomodulin sequence from a subject to provide a prognosis of the subject's ability to recover from an inflammatory condition, the kit comprising, a restriction enzyme capable of distinguishing alternate nucleotides at the polymorphism site or a labeled oligonucleotide having sufficient complementarity to the polymorphism site and capable of distinguishing said alternate nucleotides. The kit may also include one or more of the following: a package; instructions for using the kit to determine genotype; reagents such a buffers, nucleotides and enzymes. A kit as described herein may contain any combination of the following: a restriction enzyme capable of distinguishing alternate nucleotides at a thrombomodulin polymorphism site; and/or a labeled oligonucleotide having sufficient complementary to the thrombomodulin polymorphism site and capable of distinguishing said alternate nucleotides; and/or an oligonucleotide or a set of oligonucleotides suitable for amplifying a region including the thrombomodulin polymorphism site. The kit may also include one or more of the following: a package; instructions for using the kit to determine genotype; reagents such a buffers, nucleotides and enzymes; and/or containers.

The kit comprising a restriction enzyme may also comprise an oligonucleotide or a set of oligonucleotides suitable to amplify a region surrounding the polymorphism site, a polymerization agent and instructions for using the kit to determine genotype.

In accordance with another aspect of the invention, there is provided a kit for determining a genotype at a defined nucleotide position within a polymorphism site in a thrombomodulin sequence from a subject to provide a prognosis of the subject's ability to recover from an inflammatory condition, the kit comprising, in a package a restriction enzyme capable of distinguishing alternate nucleotides at the polymorphism site or a labeled oligonucleotide having sufficient complementary to the polymorphism site and capable of distinguishing said alternate nucleotides. The polymorphism site may correspond to position 5318 or position 4007 of SEQ ID NO:1.

In accordance with another aspect of the invention, oligonucleotides are provided that may be used in the identification of thrombomodulin polymorphisms in accordance with the methods described herein, the oligonucleotides are characterized in that the oligonucleotides hybridize under normal hybridization conditions with a region of one of sequences identified by SEQ ID NO:1 or its complement.

In accordance with another aspect of the invention, an oligonucleotide primer is provided comprising a portion of SEQ ID NO:1, or its complement, wherein said primer is ten to fifty-four nucleotides in length and wherein the primer specifically hybridizes to a region of SEQ ID NO:1 or its complement and is capable of specifically identifying thrombomodulin polymorphisms described herein. Alternatively, the primers may be between sixteen to twenty-four nucleotides in length.

In accordance with another aspect of the invention, methods are provided for subject screening, comprising the steps of (a) obtaining thrombomodulin sequence information from a subject, and (b) determining the identity of one or more polymorphisms in the sequence, wherein the one or more polymorphisms may be indicative of the ability of a subject to recover from an inflammatory condition.

In accordance with another aspect of the invention methods are provided for subject screening whereby the method includes the steps of (a) selecting a subject based on risk of developing an inflammatory condition or having an inflammatory condition, (b) obtaining thrombomodulin sequence information from the subject and (c) detecting the identity of one or more polymorphisms in the thrombomodulin sequence, wherein the polymorphism is indicative of the ability of a subject to recover from an inflammatory condition.

In accordance with another aspect of the invention, methods are provided for selecting a group of subjects to determine the efficacy of a candidate drug known or suspected of being useful for the treatment of an inflammatory condition, the method including determining a genotype for one or more polymorphism sites in the thrombomodulin sequence for each subject, wherein said genotype is indicative of the subject's ability to recover from the inflammatory condition and sorting subjects based on their genotype. The method may also include administering the candidate drug to the subjects or a subset of subjects and determining each subject's ability to recover from the inflammatory condition. The method may also include the additional step of comparing subject response to the candidate drug based on genotype of the subject. Response to the candidate drug may be decided by determining each subject's ability to recover from the inflammatory condition.

Risk genotypes may have at least one nucleotide selected alone or in combination from the following thrombomodulin alleles in SEQ ID NO:1:
5318 A; and
4007 C.

Risk genotype may be an indication of an increased risk of not recovering from an inflammatory condition. Subjects having one copy (heterozygotes) or two copies (homozygotes) of the risk allele (i.e. 5318 AC or 5318 AA or alternatively 4007 CT or 4007 CC or a combination thereof) are considered to have the “risk genotype” even though the degree to which the subjects risk of not recovering from an inflammatory condition increases, may be greater for homozygotes over heterozygotes.

Non-risk genotypes (protective genotypes) may be selected alone or in combination from the following thrombomodulin alleles in SEQ ID NO:1:
5318C; and
4007T.

Protective genotype may be an indication of a decreased risk of not recovering from an inflammatory condition or increase likelihood of recovery from an inflammatory condition. Subjects having two copies (homozygotes) of the protective allele (i.e. 5318 CC or 4007 TT or a combination thereof) are considered to have the “protective genotype”.

In accordance with another aspect of the invention, there is provided an oligonucleotide of about 10 to about 400 nucleotides that hybridizes specifically to a sequence contained in a human target sequence including of SEQ ID NO:1, a complementary sequence of the target sequence or RNA equivalent of the target sequence and wherein the oligonucleotide is operable in determining a risk polymorphism genotype.

In accordance with another aspect of the invention, there is provided an oligonucleotide of about 10 to about 400 nucleotides that hybridizes specifically to a sequence contained in a human target sequence including of SEQ ID NO:1, a complementary sequence of the target sequence or RNA equivalent of the target sequence and wherein said hybridization is operable in determining a risk polymorphism genotype.

In accordance with another aspect of the invention, there is provided an oligonucleotide probe selected from the group including:
(a) a probe that hybridizes under high stringency conditions to a nucleic acid molecule including SEQ ID NO:1 having a A at position 5318 but not to a nucleic acid molecule including SEQ ID NO:1 having a C at position 5318;
(b) a probe that hybridizes under high stringency conditions to a nucleic acid molecule including SEQ ID NO:1 having a C at position 5318 but not to a nucleic acid molecule including SEQ ID NO:1 having a A at position 5318;
(c) a probe that hybridizes under high stringency conditions to a nucleic acid molecule including SEQ ID NO:1 having a C at position 4007 but not to a nucleic acid molecule including SEQ ID NO:1 having a T at position 4007; and
(d) a probe that hybridizes under high stringency conditions to a nucleic acid molecule including SEQ ID NO:1 having a T at position 4007 but not to a nucleic acid molecule including SEQ ID NO:1 having a C at position 4007.

In accordance with another aspect of the invention, there is provided an array of nucleic acid molecules attached to a solid support, the array including an oligonucleotide that will hybridze to a nucleic acid molecule consisting of SEQ ID NO:1, wherein the nucleotide at position 5318 is A, under conditions in which the oligonucleotide will not substantially hybridize to a nucleic acid molecule consisting of SEQ ID NO:1 wherein the nucleotide at position 5318 is C.

In accordance with another aspect of the invention, there is provided an array of nucleic acid molecules attached to a solid support, the array including an oligonucleotide that will hybridze to a nucleic acid molecule consisting of SEQ ID NO:1, wherein the nucleotide at position 5318 is C, under conditions in which the oligonucleotide will not substantially hybridize to a nucleic acid molecule consisting of SEQ ID NO:1 wherein the nucleotide at position 5318 is A.

In accordance with another aspect of the invention, there is provided an array of nucleic acid molecules attached to a solid support, the array including an oligonucleotide that will hybridze to a nucleic acid molecule consisting of SEQ ID NO:1, wherein the nucleotide at position 4007 is C, under conditions in which the oligonucleotide will not substantially hybridize to a nucleic acid molecule consisting of SEQ ID NO:1 wherein the nucleotide at position 4007 is T.

In accordance with another aspect of the invention, there is provided an array of nucleic acid molecules attached to a solid support, the array including an oligonucleotide that will hybridze to a nucleic acid molecule consisting of SEQ ID NO:1, wherein the nucleotide at position 4007 is T, under conditions in which the oligonucleotide will not substantially hybridize to a nucleic acid molecule consisting of SEQ ID NO:1 wherein the nucleotide at position 4007 is C.

The oligonucleotides may further include one or more of the following: a detectable label; a quencher; a mobility modifier; a contiguous non-target sequence situated 5′ or 3′ to the target sequence.

In accordance with another aspect of the invention, there is provided a computer readable medium including a plurality of digitally encoded genotype correlations selected from the thrombomodulin genotype correlations in TABLE 2B, wherein each correlation of the plurality has a value representing an ability to recover from an inflammatory condition.

The above identified sequence positions refer to the sense strand of the THBD sequence as indicated. It will be obvious to a person skilled in the art that analysis could be conducted on the anti-sense strand to determine subject outcome.

### BRIEF DESCRIPTION OF THE DRAWINGS

FIG. 1 shows haplotypes and haplotype clades for thrombomodulin (THBD)

FIG. 2 shows a phylogenetic tree of THBD haplotypes generated with MEGA2 software.

FIG. 3 shows a 28 day mortality rates by THBD haplotype clade.

FIG. 4 shows a 28 day mortality rates by THBD haplotype clade in subjects with sepsis or septic shock on day one.

FIG. 5 shows 28 day mortality rates associated with THBD 5318 A and C alleles in 130 subjects with sepsis or septic shock on day one.

FIG. 6 shows a DAF of cardiovascular dysfunction by THBD 5318 A and C alleles.

FIG. 7 shows a DAF of respiratory dysfunction by THBD 5318 A and C alleles.

### DETAILED DESCRIPTION OF THE INVENTION

### 1. Definitions

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T or A, a nucleotide represented by the symbol Y may be either an C or T, a nucleotide represented by the symbol S may be either an G or C, while a nucleotide represented by the symbol R may be either an G or A.

A “polymorphic site” or “polymorphism site” or “polymorphism” or “single nucleotide polymorphism site” (SNP site) as used herein is the locus or position within a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphism sites may be at known positions within a nucleic acid sequence or may be determined to exist using the methods described herein. Polymorphisms may occur in both the coding regions and the noncoding regions (for example, promoters, enhancers and introns) of genes.

In general the term “linkage”, as used in population genetics, refers to the co-inheritance of two or more nonallelic genes due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

A “risk genotype” as used herein refers to an allelic variant (genotype) at one or more polymorphism sites within the thrombomodulin sequence described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Such “risk alleles” or “risk genotype” may be selected from positions 5318A and 4007C of SEQ ID NO: 1 (thrombomodulin).

A “clade” is a group of haplotypes that are closely related phylogenetically. For example, if haplotypes are displayed on a phylogenetic (evolutionary) tree a clade includes all haplotypes contained within the same branch.

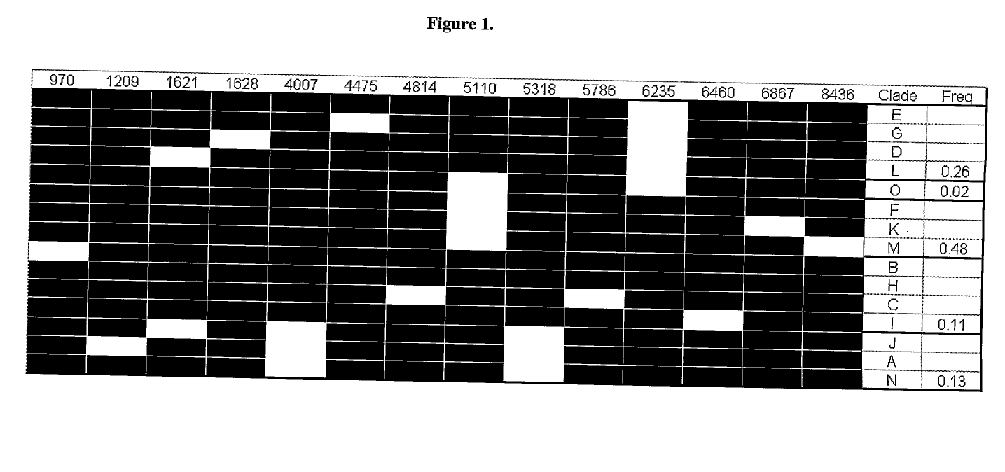

As used herein “haplotype” is a set of alleles of closely linked loci or a pattern of a set of markers along a chromosome that tend to be inherited together. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping. “Haplotypes” are shown as rows in the Table (haplotype map) represented in FIG. 1.

In general, the detection of nucleic acids in a sample and the subtypes thereof depends on the technique of specific nucleic acid hybridization in which the oligonucleotide probe is annealed under conditions of “high stringency” to nucleic acids in the sample, and the successfully annealed probes are subsequently detected (Spiegelman, S., Scientific American, Vol. 210, p. 48 (1964)). Hybridization under high stringency conditions primarily depends on the method used for hybridization. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for D′ of ≧0.8 or r²≧0.8) are potentially useful in predicting the identity of an allele of interest (i.e. associated with the condition of interest). Alternatively, a high degree of linkage disequilibrium may be represented by an absolute value for D′ of ≧0.85 or r²≧0.85 or by an absolute value for D′ of ≧0.9 or r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. For example, in the population from which the present haplotype map was created the SNP at position 5318 of SEQ. ID NO:1 was in LD with position 4007 of SEQ. ID NO:1, whereby when the genotype of 5318 is C the genotype of 4007 is T. Similarly, when the genotype of 5318 is A the genotype of 4007 is C. Accordingly, the determination of the genotype of a single locus can provide the identity of the genotype of any locus in LD therewith and the higher the degree of linkage disequilibrium the more likely that two SNPs may be used interchangeably.

Numerous sites have been identified as polymorphism sites in the thrombomodulin sequence, where those polymorphisms are linked to the polymorphism at position 5318 of SEQ. ID NO:1 and may also therefore be indicative of subject prognosis. The position 4007 of SEQ. ID NO:1 is shown to be in LD with position 5318 of SEQ. ID NO:1.

It will be appreciated by a person of skill in the art that further linked SNP sites could be determined. The haplotype for thrombomodulin can be created by assessing the SNPs of the thrombomodulin sequence in normal subjects using a program that has an expectation maximization algorithm (i.e. PHASE). A constructed haplotype of thrombomodulin may be used to find combinations of SNPs that are in linkage disequilibrium (LD) with position 5318 or position 4007 of SEQ ID NO:1. Therefore, the haplotype of an individual could be determined by genotyping other SNPs that are in LD with position 5318 or position 4007 of SEQ ID NO:1. Linked single polymorphism sites or combined polymorphism sites could also be genotyped for assessing subject prognosis.

It will be appreciated by a person of skill in the art, that the numerical designations of the positions of polymorphisms within a sequence are relative to a specific sequence and that the same positions may be assigned different numerical designations depending on the way in which the sequence is numbered and the sequence chosen, as illustrated by the alternative numbering of equivalent polymorphisms in Chao et al. (2004); Park et al. (2002); Wu et al. (2001); and Norlund et al. (1997) above. Furthermore, sequence variations within the population, such as insertions or deletions, may change the relative position and subsequently the numerical designations of particular nucleotides at and around a polymorphism site.

A representative of a Homo sapiens thrombomodulin (THBD) sequence which comprises a sequence as listed in GenBank under accession number AF495471 is found in SEQ ID NO:1. Polymorphism sites at positions 5318, 4007, 5110 and 6235 of SEQ ID NO:1 and he major and minor alleles for 5318, 4007, 5110 and 6235 of SEQ ID NO:1 (THBD sequence) are as follows:
at position 5318 the most common nucleotide (major allele) is a and the minor allele is c;
at position 4007 the most common nucleotide (major allele) is c and the minor allele is t;
at position 5110 the most common nucleotide (major allele) is a and the minor allele is g; and
at position 6235 the most common nucleotide (major allele) is a and the minor allele is g.

TABLE 1A below shows the flanking sequences for SNPs A5318C (rs3176123) and C4007T (rs1042579) of THBD along with their associated SNP locations within the sequence M and Y respectively and within the gene. Also shown in TABLE 1A is the minor allele frequency.

TABLE 1A

| | SNP | Minor | |
|---|---|---|---|
| THBD | locations in | Allele | |
| SNP | THBD | Frequency | FLANKING SEQUENCE |
| A5318C | 3′ UTR | C = 0.13 | TTACTTATTTTTGACAGTGTTGAAAATGTTCAG |
| | | | AAGGTTGCTCTAGATTGMGAGAAGAGACAAACA |
| | | | CCTCCCAGGAGACAGTTCAAGAAAGCTTCAAAC |
| | | | TG |
| | | | (SEQ ID NO: 2) |
| C4007T | Exon 1 | T = 0.16 | GCGTCTGCGCCGAGGGCTTCGCGCCCATTCCCC |
| | | | ACGAGCCGCACAGGTGCCAGATGTTTTGCAACC |
| | | | AGACTGCCTGTCCAGCCGACTGCGACCCCAACA |
| | | | CCCAGGCTAGCTGTGAGTGCCCTGAAGGCTACA |
| | | | TCCTGGACGACGGTTTCATCTGCACGGACATCG |
| | | | ACGAGTGCGAAAACGGCGGCTTCTGCTCCGGGG |
| | | | TGTGCCACAACCTCCCCGGTACCTTCGAGTGCA |
| | | | TCTGCGGGCCCGACTCGGCCCTTGYCCGCCACA |
| | | | TTGGCACCGACTGTGACTCCGGCAAGGTGGACG |
| | | | GTGGCGACAGCGGCTCTGGCGAGCCCCCGCCCA |
| | | | GCCCGACGCCCGGCTCCACCTTGACTCCTCCGG |
| | | | CCGTGGGGCTCGTGCATTCGGGCTTGCTCATAG |
| | | | GCATCTCCATCGCGAGCCTGTGCCTGGTGGTGG |
| | | | CGCTTTTGGCGGTCCTCTGCCACCTGCGCAAGA |
| | | | AGCAGGGCGCCGCCAGGGCCAAGATGGAGTACA |
| | | | AGTGCGCGGCCCCTTC |
| | | | (SEQ ID NO: 3) |

The Sequences given in TABLE 1A above and in SEQ ID NO:1 would be useful to a person of skill in the art in the design of primers and probes or other oligonucleotides for the identification of THBD SNP alleles and or genotypes as described herein.

TABLE 1B show shows genotype correlations for thrombomodulin SNPs with a value representing an ability to recover from an inflammatory condition or predicted patient outcome. However, it will be appreciated by persons of skill in the art that the Inflammatory Condition Patient Score may have a dominant/recessive relationship whereby the heterozygote provides the same score as one of the homozygotes. The relationship may also depend on the population tested.

TABLE 1B

| Position in | | | Patient Outcome |
|---|---|---|---|
| SEQ ID NO: 1 | Allele | Genotype | Score* |
| 5318 | A | AA | 0 |
| 5318 | A/C | AC | 1 |
| 5318 | C | CC | 2 |
| 4007 | C | CC | 0 |
| 4007 | C/T | CT | 1 |
| 4007 | T | TT | 2 |

*good = 2; moderate = 1; poor = 0.

An “allele” is defined as any one or more alternative forms of a given gene at a particular locus on a chromosome. Different alleles produce variation in inherited characteristics such as hair color or blood type. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene. In an individual, one form of the allele (major) may be expressed more than another form (minor). When “genes” are considered simply as segments of a nucleotide sequence, allele refers to each of the possible alternative nucleotides at a specific position in the sequence. For example, a CT polymorphism such as CCT[C/T]CCAT would have two alleles: C and T.

A “gene” is an ordered sequence of nucleotides located in a particular position on a particular chromosome that encodes a specific functional product and may include untranslated and untranscribed sequences in proximity to the coding regions. Such non-coding sequences may contain regulatory sequences needed for transcription and translation of the sequence or introns etc.

A “genotype” is defined as the genetic constitution of an organism, usually in respect to one gene or a few genes or a region of a gene relevant to a particular context (for example the genetic loci responsible for a particular phenotype). A region of a gene can be as small as a single nucleotide in the case of a single nucleotide polymorphism.

A “phenotype” is defined as the observable characters of an organism.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins”) of a nucleotide relative to a reference allele. Furthermore, it would be appreciated by a person of skill in the art, that an insertion or deletion within a given sequence could alter the relative position and therefore the position number of another polymorphism within the sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, and D) white blood cell count >12,000 per mm3 or <4,000 mm3. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Patient outcome or prognosis as used herein refers the ability of a patient to recover from an inflammatory condition. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Grain negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome or prognosis may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.”

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR) >1.5 are days that a person is alive and does not have an INR >1.5.

TABLE 2A
Brussels Organ Dysfunction Scoring System

| | Free of Organ Dysfunction | | Clinically Significant Organ Dysfunction | | |
|---|---|---|---|---|---|
| | Normal | Mild | Moderate | Severe | Extreme |
| | DAF ORGAN DYSFUNCTION SCORE | | | | |
| ORGANS | 1 | | 0 | | |
| Cardiovascular | >90 | ≦90 | ≦90 | ≦90 plus | ≦90 plus |
| Systolic BP | | Responsive | Unresponsive | pH ≦7.3 | pH ≦7.2 |
| (mmHg) | | to fluid | to fluid | | |
| Pulmonary | >400 | 400-301 | 300-201 | 200-101 | ≦100 |
| Pₐo₂/F_{I}o₂ | | | Acute lung | ARDS | Severe |
| (mmHg) | | | injury | | ARDS |
| Renal | <1.5 | 1.5-1.9 | 2.0-3.4 | 3.5-4.9 | ≧5.0 |
| Creatinine | | | | | |
| (mg/dL) | | | | | |
| Hepatic | <1.2 | 1.2-1.9 | 2.0-5.9 | 6.0-11.9 | ≧12 |
| Bilirubin | | | | | |
| (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by Chomczynski (U.S. Pat. No. 5,945,515), whereby genetic material can be extracted efficiently in as little as twenty minutes. Evans and Hugh (U.S. Pat. No. 5,989,431) describe methods for isolating DNA using a hollow membrane filter.

Once a subject's genetic sequence information has been obtained from the subject it may then be further analyzed to detect or determine the identity or genotype of one or more polymorphisms in the THBD sequence. Provided that the genetic material obtained, contains the sequence of interest. Particularly, a person may be interested in determining the THBD genotype of a subject of interest, where the genotype includes a nucleotide corresponding to position 5318 or SEQ ID NO:1 or position 4007 of SEQ ID NO:1. The sequence of interest may also include other THBD polymorphisms or may also contain some of the sequence surrounding the polymorphism of interest. Detection or determination of a nucleotide identity or the genotype of one or more single nucleotide polymorphism(s) (SNP typing), may be accomplished by any one of a number methods or assays known in the art. Many DNA typing methodologies are useful for allelic discrimination and detection of SNPs. Furthermore, the products of allelic discrimination reactions or assays may be detected by one or more detection methods. The majority of SNP genotyping reactions or assays can be assigned to one of four broad groups (allele specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, allele specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and allelic discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, allele specific extensions, or simple PCR amplification) are useful in allelic discrimination reactions. For example, in mini sequencing a primer anneals to its target DNA immediately upstream of the SNP and is extended with a single nucleotide complementary to the polymorphic site. Where the nucleotide is not complementary no extension occurs.

Oligonucleotide ligation assays require two allele specific probes and one common ligation probe per SNP. The common ligation probe hybridizes adjacent to an allele specific probe and when there is a perfect match of the appropriate allele specific probe the ligase joins both allele specific and the common probes. Where there is not a perfect match the ligase is unable to join the allelic specific and common probes.

Alternatively, an invasive cleavage method requires an oligonucleotide called an invader probe and allele specific probes to anneal to the target DNA with an overlap of one nucleotide. When the allele specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the major allele and the other to the minor allele. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragment. Mismatched probes are displaced without fragmentation. The genotype of a sample is determined by measuring the signal intensity of the two different dyes.

It will be appreciated that numerous other methods for allelic discrimination and detection are known in the art and some of which are described in further detail below. It will also be appreciated that reactions such as arrayed primer extension mini sequencing, tag microarrays and allelic specific extension could be performed on a microarray. One such array based genotyping platform is the microsphere based tag-it high throughput genotyping array (Bortolin S. et al. Clinical Chemistry (2004) 50(11): 2028-36). This method amplifies genomic DNA by PCR followed by allele specific primer extension with universally tagged genotyping primers. The products are then sorted on a Tag-It array and detected using the Luminex xMAP system.

SNP typing methods may include but are not limited to the following:
Restriction Fragment Length Polymorphism (RFLP) strategy—An RFLP gel-based analysis can be used to distinguish between alleles at polymorphic sites within a gene. Briefly, a short segment of DNA (typically several hundred base pairs) is amplified by PCR. Where possible, a specific restriction endonuclease is chosen that cuts the short DNA segment when one variant allele is present but does not cut the short DNA segment when the other allele variant is present. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the initial DNA sample had the allele, which could be cut by the chosen restriction endonuclease. In contrast, if only one higher molecular weight band is observed (at the molecular weight of the PCR product) then the initial DNA sample had the allele variant that could not be cut by the chosen restriction endonuclease. Finally, if both the higher molecular weight band and the two lower molecular weight bands are visible then the initial DNA sample contained both alleles, and therefore the subject was heterozygous for this single nucleotide polymorphism;
Sequencing—For example the Maxam-Gilbert technique for sequencing (Maxam A M. and Gilbert W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by Sanger et al. (Sanger et al. Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxy-nucleotides have been used to sequence encephalomyocarditis virus RNA (Zimmem D. and Kaesberg P. Proc. Natl. Acad. Sci. USA (1978) 75(9):4257-4261). Mills D R. and Kramer F R. (Proc. Natl. Acad. Sci. USA (1979) 76(5):2232-2235) describe the use of Q.beta. replicase and the nucleotide analog inosine for sequencing RNA in a chain-termination mechanism. Direct chemical methods for sequencing RNA are also known (Peattie D A. Proc. Natl. Acad. Sci. USA (1979) 76(4):1760-1764). Other methods include those of Donis-Keller et al. (1977, Nucl. Acids Res. 4:2527-2538), Simoncsits A. et al. (Nature (1977) 269(5631):833-836), Axelrod V D. et al. (Nucl. Acids Res. (1978) 5(10):3549-3563), and Kramer F R. and Mills D R. (Proc. Natl. Acad. Sci. USA (1978) 75(11):5334-5338, which are incorporated herein by reference). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;
Hybridization methods for the identification of SNPs are described in the U.S. Pat. Nos. 6,270,961 & 6,025,136;
A template-directed dye-terminator incorporation with fluorescent polarization-detection (TDI-FP) method is described by FREEMAN B D. et al. (J Mol Diagnostics (2002) 4(4):209-215) is described for large scale screening;
Oligonucleotide ligation assay (OLA)—is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2): 131-40);
Ligation-Rolling Circle Amplification (L-RCA) has also been successfully used for genotyping single nucleotide polymorphisms as described in QI X. et al. Nucleic Acids Res (2001) 29(22):E116;
5′ nuclease assay has also been successfully used for genotyping single nucleotide polymorphisms (AYDIN A. et al. Biotechniques (2001) (4):920-2, 924, 926-8.);
Polymerase proofreading methods are used to determine SNPs identities, as described in WO 0181631;
Detection of single base pair DNA mutations by enzyme-amplified electronic transduction is described in PATOLSKY F et al. Nat. Biotech. (2001) 19(3):253-257;
Gene chip technologies are also known for single nucleotide polymorphism discrimination whereby numerous polymorphisms may be tested for simultaneously on a single array (EP 1120646 and Gilles P N. et al. Nat. Biotechnology (1999) 17(4):365-70);
Matrix assisted laser desorption ionization time of flight (MALDI-TOF) mass spectroscopy is also useful in the genotyping single nucleotide polymorphisms through the analysis of microsequencing products (Haff L A. and Smirnov I P. Nucleic Acids Res. (1997) 25(18):3749-50; Haff L A. and Smirnov I P. Genome Res. (1997) 7:378-388; Sun X. et al. Nucleic Acids Res. (2000) 28 e68; Braun A. et al. Clin. Chem. (1997)43:1151-1158; Little D P. et al. Eur. J. Clin. Chem. Clin. Biochem. (1997) 35:545-548; Fei Z. et al. Nucleic Acids Res. (2000) 26:2827-2828; and Blondal T. et al. Nucleic Acids Res. (2003) 31(24):e155); or
Allele specific PCR methods have also been successfully used for genotyping single nucleotide polymorphisms (Hawkins J R. et al. Hum Mutat (2002) 19(5):543-553).

Alternatively, if a subject's sequence data is already known, then obtaining may involve retrieval of the subjects nucleic acid sequence data from a database, followed by determining or detecting the identity of a nucleic acid or genotype at a polymorphism site by reading the subject's nucleic acid sequence at the polymorphic site.

Once the identity of a polymorphism(s) is determined or detected an indication may be obtained as to subject outcome or prognosis or ability of a subject recover from an inflammatory condition based on the genotype (the nucleotide at the position) of the polymorphism of interest. In the present invention, polymorphisms in thrombomodulin (THBD) sequence, are used to obtain a prognosis or to make a determination regarding ability of the subject to recover from the inflammatory condition. Methods for determining a subject's prognosis or for subject screening may be useful to determine the ability of a subject to recover from an inflammatory condition. Alternatively, single polymorphism sites or combined polymorphism sites may be used as an indication of a subject's ability to recover from an inflammatory condition, if they are linked to a polymorphism determined to be indicative of a subject's ability to recover from an inflammatory condition. The method may further comprise comparing the genotype determined for a polymorphism with known genotypes, which are indicative of a prognosis for recovery from the same inflammatory condition as for the subject or another inflammatory condition. Accordingly, a decision regarding the subject's ability to recover may be from an inflammatory condition may be made based on the genotype determined for the polymorphism site.

Once subject outcome or a prognosis is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care.

An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more single nucleotide polymorphism sites in THBD sequence. Also, as previously described the sequence identity of one or more single nucleotide polymorphisms in THBD sequence of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

Clinical Phenotype

The primary outcome variable was survival to hospital discharge. Secondary outcome variables were days alive and free of cardiovascular, respiratory, renal, hepatic, hematologic, and neurologic organ system failure as well as days alive and free of SIRS (Systemic Inflammatory Response Syndrome), occurrence of sepsis, and occurrence of septic shock. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count >11,000/μL) (Anonymous. Critical Care Medicine (1992) 20(6):864-74). Subjects were included in this cohort on the calendar day on which the SIRS criteria were met.

A subject's baseline demographics that were recorded included age, gender, whether medical or surgical diagnosis for admission (according to APACHE III diagnostic codes (KNAUS W A et al. Chest (1991) 100(6):1619-36)), and admission APACHE II score. The following additional data were recorded for each 24 hour period (8 am to 8 am) for 28 days to evaluate organ dysfunction, SIRS, sepsis, and septic shock.

Clinically significant organ dysfunction for each organ system was defined as present during a 24 hour period if there was evidence of at least moderate organ dysfunction using the Brussels criteria (TABLE 2A) (RUSSELL J A et al. Critical Care Medicine (2000) 28(10):3405-11). Because data were not always available during each 24 hour period for each organ dysfunction variable, we used the “carry forward” assumption as defined previously (Anonymous. New England Journal of Medicine (2000) 342(18):1301-8). Briefly, for any 24 hour period in which there was no measurement of a variable, we carried forward the “present” or “absent” criteria from the previous 24 hour period. If any variable was never measured, it was assumed to be normal.

To further evaluate cardiovascular, respiratory, and renal function we also recorded, during each 24-hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis). In addition, severity of respiratory dysfunction was assessed, by measuring the occurrence of acute lung injury at the time of meeting the inclusion criteria. Acute lung injury was defined as having a PaO₂/FiO₂ ratio <300, diffuse infiltrates pattern on chest radiograph, and a CVP <18 mm Hg.

To assess duration of organ dysfunction and to correct organ dysfunction scoring for deaths in the 28-day observation period, calculations were made of days alive and free of organ dysfunction (DAF) as previously reported (BERNARD G R et al. New England Journal of Medicine (1997) 336(13):912-8). Briefly, during each 24-hour period for each variable, DAF was scored as 1 if the subject was alive and free of organ dysfunction (normal or mild organ dysfunction, Table 2A). DAF was scored as 0 if the subject had organ dysfunction (moderate, severe, or extreme) or was not alive during that 24-hour period. Each of the 28 days after ICU admission was scored in each subject in this fashion. Thus, the lowest score possible for each variable was zero and the highest score possible was 28. A low score is indicative of more organ dysfunction as there would be fewer days alive and free of organ dysfunction.

Similarly, days alive and free of SIRS (DAF SIRS) were calculated. Each of the four SIRS criteria were recorded as present or absent during each 24 hour period. Presence of SIRS during each 24 hour period was defined by having at least 2 of the 4 SIRS criteria. Sepsis was defined as present during a 24 hour period by having at least two of four SIRS criteria and having a known or suspected infection during the 24 hour period (Anonymous. Critical Care Medicine (1992) 20(6):864-74). Cultures that were judged to be positive due to contamination or colonization were excluded. Septic shock was defined as presence of sepsis plus presence of hypotension (systolic blood pressure <90 mmHg or need for vasopressor agents) during the same 24 hour period.

Microbiology

Microbiological cultures were taken for any patients who were suspected of having an infection. As this is a cohort of critically ill patients with SIRS, most patients had cultures taken. Positive cultures that were suspected of having been contaminated or colonized were excluded. Positive cultures that were deemed to clinically be clinically irrelevant were also excluded. Cultures were categorized as gram positive, gram negative, fungal or other. The sources of the cultures were respiratory, gastrointestinal, skin, soft tissues or wounds, genitourinary, or endovascular.

Haplotypes and Selection of htSNPs

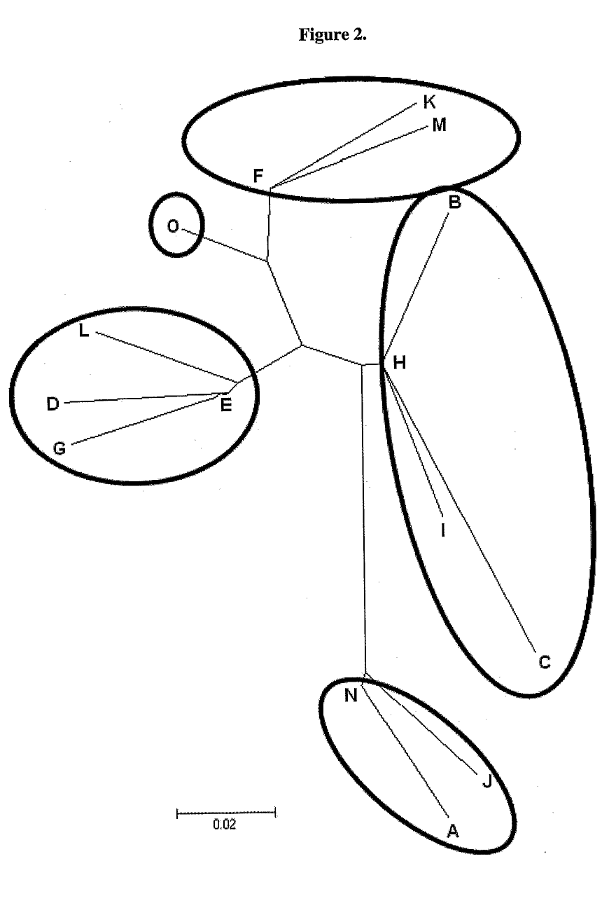

Using unphased Caucasian genotypic data (from the Coriell registry pga.mbt.washington.edu (RIEDER M J et al. SeattleSNPs. NHLBI Program for Genomic Applications, UW-FHCRC, Seattle, Wash. (2001)) haplotypes were inferred using PHASE (STEPHENS M. et al. Am J Hum Genet (2001) 68:978-89) software (FIG. 1). MEGA 2 (KUMAR S. et al. (2001) 17:1244-5) was then used to infer a phylogenetic tree to identify major haplotype clades for THBD (FIG. 2). Haplotypes were sorted according to the phylogenetic tree and haplotype structure was inspected to choose haplotype tag SNPs (htSNPs) (JOHNSON G C. et al. Nat Genet (2001) 29:233-7; and GABRIEL S B. et al. Science (2002) 296:2225-9). Three htSNPs were chosen that identified major haplotype clades of THBD in Caucasians were chosen. The first SNP was a G-to-A transition at nucleotide 5110 relative to the start transcription site (rs1042580), the second SNP was an A-to-C transversion at nucleotide 5318 (rs3176123), and the third htSNP was an A-to-G transition at nucleotide 6235 relative to the start transcription site (rs1962) (NCBI Thrombomodulin accession number AF495471) (SEQ ID NO:1). These SNPs were then genotyped in our subject cohort to define haplotypes and haplotype clades. “Tag” SNPs (tSNPs) or “haplotype tag” SNPs (htSNPs) can be selected to uniquely define a clade and serve as markers for all SNPs within haplotypes of the clade.

Blood Collection/Processing Genotyping

The buffy coat was extracted from whole blood and samples transferred into 1.5 ml cryotubes and stored at −80° C. DNA was extracted from the buffy coat of peripheral blood samples using a QIAamp DNA Blood Maxi Kit (Qiagen™). The genotypic analysis was performed in a blinded fashion, without clinical information. Polymorphisms were genotyped using a real time polymerase chain reaction (PCR) using specific fluorescence-labeled hybridization probes in the ABI Prism 7900 HT Sequence Detection System (Applied Biosystems, Inc.—Livak K J. (1999) Genet Anal 14:143-9). Briefly, the ABI Prism 7900HT uses a 5′ Nuclease Assay in which an allele-specific probe labeled with a fluorogenic reporter dye and a fluorogenic quencher is included in the PCR reaction. The probe is cleaved by the 5′ nuclease activity of Taq DNA polymerase if the probe target is being amplified, freeing the reporter dye and causing an increase in specific fluorescence intensity. Mismatched probes are not cleaved efficiently and thus do not contribute appreciably to the final fluorescent signal. An increase in a specific dye fluorescence indicates homozygosity for the dye-specific allele. An increase in both signals indicated heterozygosity. DNA from lymphocyte cell lines obtained from the Coriell Cell Repository was used to ensure the accuracy of the genotyping. The genotype of these cell lines at G5110A, A5218C and A6235 was determined using the ABI Prism 7900HT Sequence Detection system and compared to the genotype of the same cell lines determined by direct sequencing, given at www.pga.mbt.washington.edu (SeattleSNPs 2003, posting date. Thrombomodulin. SeattleSNPs. NHLBI Programs for Genomic Applications. UW-FHCRC. [Online.]).

Data Collection

Data was recorded for 28 days or until hospital discharge. Raw clinical and laboratory variables were recorded using the worst or most abnormal variable for each 24 hour period with the exception of Glasgow Coma Score, where the best possible score for each 24 hour period was recorded. Missing data on the date of admission was assigned a normal value and missing data after the day one was substituted by carrying forward the previous day's value. Demographic and microbiologic data were recorded. When data collection for each subject was complete, all subject identifiers were removed from all records and the subject file was assigned a unique random number that was cross referenced with the blood samples. The completed raw data file was converted to calculated descriptive and severity of illness scores using standard definitions (i.e. APACHE II and Days alive and free of organ dysfunction calculated using the Brussels criteria).

A chi-squared test was used to test for an association between 28-day mortality and haplotype clades. This initial analysis identified the A/C/A haplotype clade as being distinct from all other clades. For subsequent analysis differences in clinical outcomes were compared between the A/C/A haplotype clade versus all other haplotypes combined. Rates of dichotomous outcomes (28-day mortality, sepsis and shock at onset of SIRS) were compared between the 2 groups of haplotype clades using a chi-squared test. Differences in continuous outcome variables between the A/C/A haplotype clade and all other haplotype clades were tested using ANOVA. Baseline descriptive characteristics were compared using chi-squared test and ANOVA where appropriate. 28-day mortality was further compared between the A/C/A haplotype clade and all other haplotype clades while adjusting for other confounders (age, sec, and medical vs. surgical diagnosis) using a Cox regression analysis in addition to a Kaplan-Meier analysis.

Statistical Analysis

We used a cohort study design. Rates of dichotomous outcomes (28-day mortality, sepsis and shock at onset of SIRS) were compared between haplotype clades using a chi-squared test, assuming a dominant model of inheritance. Differences in continuous outcome variables between haplotype clades were tested using ANOVA. 28-day mortality was further compared between haplotype clades while adjusting for other confounders (age, sex, and medical vs. surgical diagnosis) using a Cox regression model, together with Kaplan-Meier analysis. Haplotype clade relative risk was calculated. This analysis was performed in the entire cohort, and subsequently in sub-groups of subjects who had sepsis at onset of SIRS, and subjects who had septic shock at onset of SIRS. Genotype distributions were tested for Hardy-Weinberg equilibrium (GUO S W. and THOMPSON E A. (1992) 48:361-72). We report the mean and 95% confidence intervals. Statistical significance was set at p<0.05. The data was analyzed using SPSS 11.5 for Windows™ and SigmaStat 3.0 software (SPSS Inc, Chicago, Ill., 2003).

### 3. EXAMPLES

700 consecutive critically ill patients admitted to the ICU of St. Paul's Hospital were screened for inclusion. Of these, 600 patients (94%) met the inclusion criteria of having at least two our of four SIRS criteria. From this group, 223 patients were Caucasian and were successfully genotyped and used in our final cohort for analysis.

### Example 1

### Thrombomodulin Haplotype Analysis

Haplotype Clade Deduction

It was possible to infer haplotypes from complete sequencing of THBD for 23 Caucasians in the Coriell Cell Repository (2003, posting date. Thrombomodulin. SeattleSNPs. NHLBI Programs for Genomic Applications. UW-FHCRC. [Online.]) using PHASE software (Stephens M. et al. (2001) A new statistical method for haplotype reconstruction from population data. Am J Respir Crit. Care Med 68:978-89.), and identified two major haplotype clades using MEGA2 software (Kumar S. et al. (2001) MEGA2: molecular evolutionary genetics analysis software. Bioinformatics 17:1244-5) (FIGS. 1 and 2). These 5 clades could be resolved by genotyping three htSNPs: G5110A, A5318C and A6235G, in our 223 patient cohort. The 5110G/5318A/6235A (G/A/A) haplotype clade occurred with a frequency of 36.3%, the A/A/A haplotype clade occurred with a frequency of 22.4%, and a/A/G haplotype clade occurred with a frequency of 21.5%, the A/C/A haplotype clade occurred with a frequency of 18.4%, and the G/A/G haplotype clade occurred with a frequency of 1.3%. The genotypes of all three htSNPs were similar to frequencies deduced from other available Caucasian data (2003, posting date. Thrombomodulin. SeattleSNPs. NHLBI Programs for Genomic Applications. UW-FHCRC. [Online.]) and were in Hardy-Weinberg equilibrium (Table 3) (Guo S W. and Thompson E A. (1992) Performing the exact test of Hardy-Weinberg proportion for multiple alleles. Biometrics 48:361-72).

TABLE 3
Genotype Frequencies and Allele Frequencies
for three htSNPs of thrombomodulin in a Cohort
of 223 Critically Ill Adults who had SIRS

| | Genotype Frequencies | | | Allele Frequencies | | p values* |
|---|---|---|---|---|---|---|
| | GG | GA | AA | G | A | |
| G5110A | 17% | 42% | 41% | 38% | 62% | 0.119 |
| | AA | AC | CC | A | C | |
| A5318C | 67% | 29% | 4% | 82% | 18% | 0.514 |
| | AA | AG | GG | A | G | |
| A6235G | 62% | 31% | 7% | 77% | 23% | 0.086 |

*exact test of Guo and Thompson to test for Hardy-Weinberg equilibrium

For the 223 successfully genotyped individuals of the cohort of Caucasian patients who had at least 2 of 4 SIRS criteria, no haplotype clade of THBD was significantly associated with a difference in age, gender or severity of illness at the time of admission to the study (as estimated by the APACHE II score) (Table 4).

TABLE 4
Baseline Characteristics of 223 critically ill patients
with SIRS by thrombomodulin haplotype clade

| | | | | Diagnosis for | |
|---|---|---|---|---|---|
| Haplotype | | Mean | Gender | admission | Mean |
| Clade | Frequency | Age | (% Male) | (% Surgical) | APACHE II |
| G/A/A | 36% | 59 | 60% | 26% | 18 |
| A/C/A | 18% | 59 | 61% | 44% | 19 |
| A/A/A | 22% | 59 | 69% | 23% | 20 |
| G/A/G | 1% | 69 | 50% | 17% | 19 |
| A/A/G | 22% | 61 | 68% | 33% | 20 |
| p | | NS | NS | 0.02 | NS |

By chance, the A/C/A haplotype clade was associated with a higher proportion of surgical diagnoses for admission to the ICU (Table 5).

TABLE 5
Cox Proportional Hazard Analysis - Hazard Ratios for Mortality

| Covariate | Hazard Ratio | 95% CI | p |
|---|---|---|---|
| Female sex | 0.63 | 0.41-0.98 | 0.04 |
| Age | 1.00 | 0.99-1.02 | 0.45 |
| Surgical Diagnosis | 0.77 | 0.50-1.17 | 0.21 |
| G/A/A, A/A/A, | 1.95 | 1.05-3.57 | 0.03 |
| G/A/G, or A/A/G | | | |

The A/C/A haplotype is the reference or “protective” group, leading indicating that individuals with any of the “risk” haplotypes (G/A/A, A/A/A, G/A/G, or A/A/G) are 1.95 times more likely to have a poor outcome than individuals with the ACA haplotype after adjusting for gender, age, and surgical diagnosis. The alternative way to describe the effect is to say that individuals with the ACA haplotype are 1.95 times more likely to survive or have a good outcome than individuals with all other haplotypes. The overall p-value (of the model) ˜0.03, while the p-value for relative risk (CPH regression coefficient) was ˜0.042. Accordingly, haplotypes can lead to a more powerful association test as compared to alleles or genotype.

### Example 2

### Allele Patient Outcome

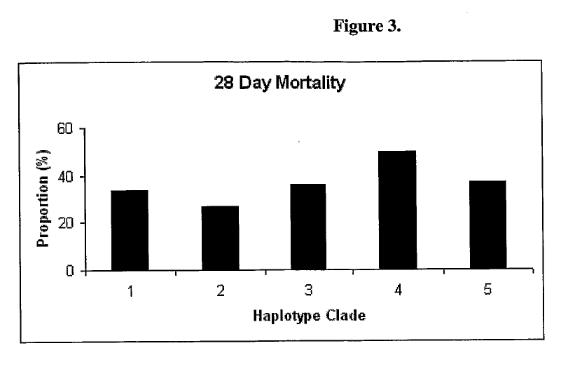

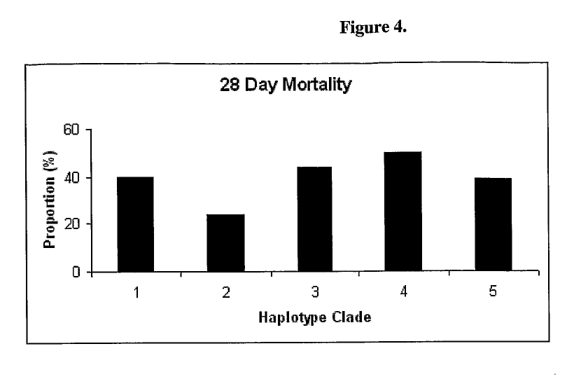

Upon preliminary analysis by ANOVA, the 5318 C allele appeared to be associated with a lower rate of 28-day mortality than the 5318 A allele (FIG. 3). This trend was stronger in patients who had sepsis or septic shock at the time they were admitted to the study (FIG. 4). We subsequently chose to compare the 5318 A allele which was associated with increased rates of 28-day mortality with the 5318 C allele. Further analysis was limited to the 130 patients who had sepsis or septic shock at the time they were admitted to the study. The average APACHE II score of these patients was 21.4±7.9. There was no difference between clades in the proportion of medical vs. surgical diagnoses in this subgroup of patients.

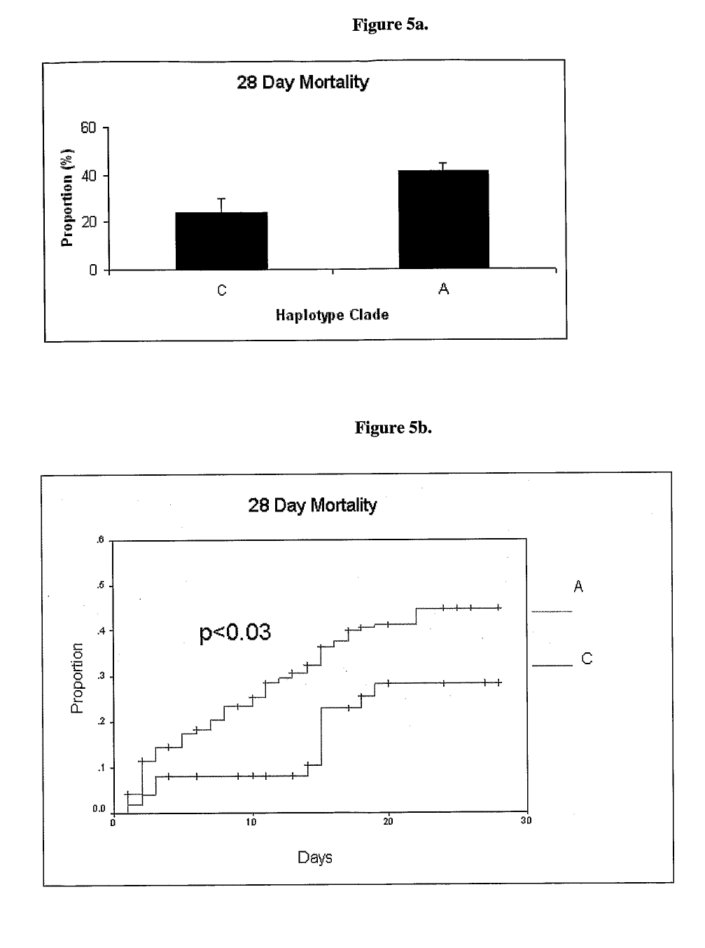

In patients who had sepsis or septic shock at the time they were admitted to the study, the 5318 A allele was associated with significantly greater 28-day mortality than the 5318 C allele (p=0.03) (FIG. 5a). Kaplan-Meier analysis of 28-day mortality verified that the 5318 A allele was significantly associated with increased rates of mortality over the entire 28-day observation period (p<0-0.03) (FIG. 5b). A Cox multiple regression model demonstrated that the 5318 A allele was an independent predictor of mortality after adjusting for other predictors of survival (age, sex, medical vs surgical diagnosis at admission) (p<0.03) (Table 4).

The 5318 A allele was associated with a more vigorous inflammatory response. In our entire 223 patient cohort, the 5318 A allele was associated with fewer DAF of 4 of 4 (20.6 days for the 5318 A allele vs. 23.1 days for the 5318 C allele, p=0.05), 3 of 4 (20.3 days for the 5318 A allele vs. 22.7 days for the 5318 C allele, p=0.06) and 2 of 4 SIRS criteria (19.9 days for the 5318 A allele clades vs. 22.4 days for the 5318 C allele, p=0.05). In the subgroup of 130 patients who had sepsis or septic shock upon admission to the study the 5318 A allele was even more strongly associated with fewer DAF of 4 of 4 (20.0 days for the 5318 A allele vs. 23.9 days for the 5318 C allele, p=0.01), 3 of 4 (19.7 days for the 5318 A allele clades vs. 23.1 days for the 5318 C allele, p=0.02) and 2 of 4 SIRS criteria (19.1 days for the 5318 A allele clades vs 23.0 days for the 5318 C allele, p=0.01).

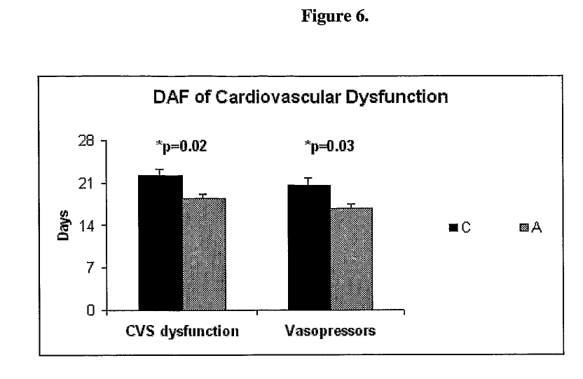

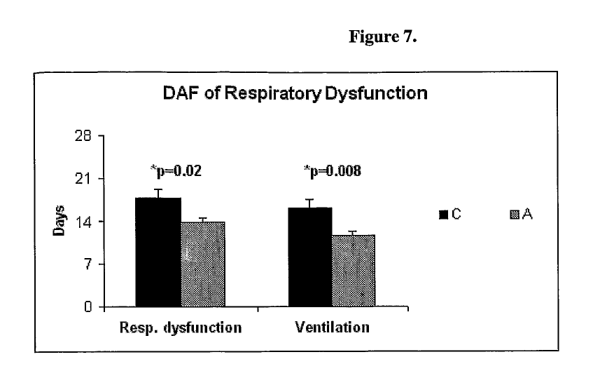

The 5318 A allele was associated with fewer days alive and free of multiple-system organ failure. The 5318 C allele was significantly associated with fewer DAF of cardiovascular failure (p=0.02), and the need for more cardiovascular support as measured by fewer DAF of vasopressors (p=0.03) (FIG. 6). The 5318 A allele was associated with fewer DAF of respiratory failure (p=0.02) and fewer DAF of ventilation (p=0.008) (FIG. 7). The 5318 A allele was also associated with fewer DAF of hematologic system failure (23.8 days for the 5318 A allele vs. 26.5 days for the 5318 C allele, p=0.04) fewer DAF of neurologic dysfunction (18.4 for the 5318 A allele vs. 22.1 days for the 5318 C allele, p=0.02), and fewer DAF of hepatic dysfunction (18.1 days for the 5318 A allele vs. 21.6 days for the 5318 C allele, p=0.04).

When analyzed individually, there was no significant association between the htSNPs G5110A, A5318C, or A6235G and 28-day mortality or multiple system organ failure.

Clinical Implications

Subjects with sepsis, severe sepsis or SIRS may be genotyped to assess their thrombomodulin 5318 and 4007 genotypes or the genotypes of polymorphism sites in linkage disequilibrium with these SNPs. Subjects could then be classified by genotype into a risk category regarding their unique risk of death by genotype.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for obtaining a prognosis for a subject having, or at risk of developing, an inflammatory condition, the method comprising determining a genotype of said subject which includes one or more polymorphic sites in the subject's thrombomodulin sequence, wherein said genotype is indicative of an ability of the subject to recover from the inflammatory condition.

2. The method of claim 1, wherein the polymorphic site is at position 5318 of SEQ ID NO:1 or at a polymorphic site in linkage disequilibrium thereto.

3. The method of claim 2, wherein the polymorphic site in linkage disequilibrium with position 5318 corresponds to position 4007 of SEQ ID NO:1.

4. The method of claim 2, wherein the polymorphic site in linkage disequilibrium with position 5318 has a D′ value of ≧0.8 or r² value ≧0.8.

5. The method of claim 1, further comprising comparing the genotype so determined with known genotypes which are known to be indicative of a prognosis for recovery from:
(i) the subject's type of inflammatory condition; or
(ii) another inflammatory condition.

6. The method of claim 2, further comprising determining the thrombomodulin sequence information for the subject.

7. The method of claim 2, wherein said determining of genotype is performed on a nucleic acid sample from the subject.

8. The method of claim 7, further comprising obtaining a nucleic acid sample from the subject.

9. The method of claim 1, wherein said determining of genotype comprises one or more of:
(a) restriction fragment length analysis;
(b) sequencing;
(c) hybridization;
(d) oligonucleotide ligation assay;
(e) ligation rolling circle amplification;
(f) 5′ nuclease assay;
(g) polymerase proofreading methods;
(h) allele specific PCR; and
(i) reading sequence data.

10. The method of claim 1, wherein a risk genotype for the subject is indicative of a decreased likelihood of recovery from an inflammatory condition or an increased risk of having a poor outcome.

11. The method of claim 10, wherein the subject is critically ill and the risk genotype is indicative of a prognosis of severe cardiovascular or respiratory dysfunction.

12. The method of claim 10, wherein the risk genotype comprises at least one A nucleotide at position 5318 or at least one C nucleotide at position 4007 of SEQ ID NO:1.

13. The method of claim 1, wherein the protective genotype of the subject is indicative of an increased likelihood of recovery from an inflammatory condition.

14. The method of claim 13, wherein the subject is critically ill and the protective genotype is indicative of a prognosis of less severe cardiovascular or respiratory dysfunction.

15. The method of claim 13, wherein the protective genotype is homozygous for the C nucleotide at position 5318 or homozygous for the T nucleotide at position 4007 of SEQ ID NO:1.

16. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for patients undergoing major surgery or dialysis, patients who are immunocompromised, patients on immunosuppressive agents, patients with HIV/AIDS, patients with suspected endocarditis, patients with fever, patients with fever of unknown origin, patients with cystic fibrosis, patients with diabetes mellitus, patients with chronic renal failure, patients with bronchiectasis, patients with chronic obstructive lung disease, chronic bronchitis, emphysema, or asthma, patients with febrile neutropenia, patients with meningitis, patients with septic arthritis, patients with urinary tract infection, patients with necrotizing fasciitis, patients with other suspected Group A streptococcus infection, patients who have had a splenectomy, patients with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

17. The method of claim 1, wherein the inflammatory condition is SIRS.

18-23. (canceled)

24. A method for selecting a group of subjects for determining the efficacy of a candidate drug known or suspected of being useful for the treatment of an inflammatory condition, the method comprising determining a genotype for one or more polymorphic sites in the thrombomodulin sequence for each subject, wherein said genotype is indicative of the subject's ability to recover from the inflammatory condition and sorting subjects based on their genotype.

25. The method of claim 24 further comprising, administering the candidate drug to the subjects or a subset of subjects and determining each subject's ability to recover from the inflammatory condition.

26. The method of claim 25, further comprising comparing subject response to the candidate drug based on genotype of the subject.

27. An oligonucleotide of about 10 to about 400 nucleotides that hybridizes specifically to a sequence contained in a human target sequence consisting of SEQ ID NO:1, a complementary sequence of the target sequence or RNA equivalent of the target sequence and wherein the oligonucleotide is operable in determining a polymorphism genotype.

28. (canceled)

29. An oligonucleotide probe selected from the group consisting of:
(a) a probe that hybridizes under high stringency conditions to a nucleic acid molecule comprising SEQ ID NO:1 having a A at position 5318 but not to a nucleic acid molecule comprising SEQ ID NO:1 having a C at position 5318;
(b) a probe that hybridizes under high stringency conditions to a nucleic acid molecule comprising SEQ ID NO:1 having a C at position 5318 but not to a nucleic acid molecule comprising SEQ ID NO:1 having a A at position 5318;
(c) a probe that hybridizes under high stringency conditions to a nucleic acid molecule comprising SEQ ID NO:1 having a C at position 4007 but not to a nucleic acid molecule comprising SEQ ID NO:1 having a T at position 4007; and
(d) a probe that hybridizes under high stringency conditions to a nucleic acid molecule comprising SEQ ID NO:1 having a T at position 4007 but not to a nucleic acid molecule comprising SEQ ID NO:1 having a C at position 4007.

30. An array of nucleic acid molecules attached to a solid support, the array comprising one or more oligonucleotides selected from the following:
(a) an oligonucleotide that will hybridize to a nucleic acid molecule consisting of SEQ ID NO:1, wherein the nucleotide at position 5318 is A, under conditions in which the oligonucleotide will not substantially hybridize to a nucleic acid molecule consisting of SEQ ID NO:1 wherein the nucleotide at position 5318 is C;
(b) an oligonucleotide that will hybridize to a nucleic acid molecule consisting of SEQ ID NO:1, wherein the nucleotide at position 5318 is C, under conditions in which the oligonucleotide will not substantially hybridize to a nucleic acid molecule consisting of SEQ ID NO:1 wherein the nucleotide at position 5318 is A;
(c) an oligonucleotide that will hybridize to a nucleic acid molecule consisting of SEQ ID NO:1, wherein the nucleotide at position 4007 is C, under conditions in which the oligonucleotide will not substantially hybridize to a nucleic acid molecule consisting of SEQ ID NO:1 wherein the nucleotide at position 4007 is T; and
(d) an oligonucleotide that will hybridize to a nucleic acid molecule consisting of SEQ ID NO:1, wherein the nucleotide at position 4007 is T, under conditions in which the oligonucleotide will not substantially hybridize to a nucleic acid molecule consisting of SEQ ID NO:1 wherein the nucleotide at position 4007 is C.

31-33. (canceled)

34. An oligonucleotide of claim 27 further comprising one or more of the following: a detectable label; a quencher; a mobility modifier; a contiguous non-target sequence situated 5′ or 3′ to the target sequence.

35. A computer readable medium comprising a plurality of digitally encoded genotype correlations selected from the thrombomodulin genotype correlations in TABLE 2B, wherein each correlation of the plurality has a value representing an ability to recover from an inflammatory condition.

36. An oligonucleotide of claim 29 further comprising one or more of the following: a detectable label; a quencher; a mobility modifier; a contiguous non-target sequence situated 5′ or 3′ to the target sequence
